(19) United States
(12) Patent Application Publication
(10) Pub. No.: US 2010/0106122 A1
(43) Pub. Date: Apr. 29, 2010

(54) FLUID EXTRACTING WOUND DRESSING

(75) Inventor: Leslie CLEMENS, Schaumburg, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: SESSIONS PHARMACEUTICALS INC., Burr Ridge, IL (US)

(21) Appl. No.: 12/605,954

(22) Filed: Oct. 26, 2009

Related U.S. Application Data
(60) Provisional application No. 61/108,746, filed on Oct. 27, 2008.

(51) Int. Cl. A61F 13/53

(52) U.S. Cl. 604/369

(57) ABSTRACT

A wound dressing including a composite body of predetermined length adapted for insertion into a wound cavity. The composite body includes a hydrophilic foam matrix. A first layer of polymer-based mesh is disposed at a first position within the foam matrix and at least a second layer of polymer-based mesh is disposed at a second position within the foam matrix in spaced apart opposing relation to the first layer of polymer-based mesh. The first layer of polymer-based mesh and the second layer of polymer-based mesh are in sandwiching relation to a foam core. The wound dressing provides among other advantages strength and substantially enhanced wicking action.

### RELATED APPLICATIONS

This application claims the benefit of, and priority from, U.S. Provisional Application No. 61/108,746, filed Oct. 27, 2008, the contents of which are incorporated herein by reference in their entirety.

### TECHNICAL FIELD

The present invention relates to wound dressings, and more particularly to wound dressing suitable for use in managing deep or connected or complicated wounds such as cavity wounds that include undermining, fistulas, or tunneling wounds.

### BACKGROUND

In the management of deep or tunneling wounds, it is desirable to temporarily fill and keep such wounds open while promoting gradual closure from the distal end to the proximal opening, and free from external contamination, or slough or debris from the wound. It is further desirable to keep such wounds sterile. It is further desirable to avoid substantial accumulation of wound exudates at the wound situs, such as blood, pus, and other wound fluids, since the presence of accumulated exudates may promote the growth of bacteria or other microorganisms which delay the healing process.

Wound dressings comprising gauze, hydrogel, alginates, or foam-based absorbent materials have long been used to treat such wounds. After insertion into the wound, these dressings may expand as they absorb the fluid exudate and are subsequently removed and replaced with a new dressing in accordance with known wound care protocols. These dressings also may include antiseptics and/or anti-bacterial agents such as silver-based compositions and the like.

### SUMMARY OF THE INVENTION

The present invention provides advantages and alternatives over the prior art by providing an elongate foam-based wound dressing incorporating at least two layers of mesh disposed in spaced relation from one another across the thickness dimension of the dressing.

In accordance with one aspect, the present invention provides a wound dressing including a composite body of predetermined length adapted for insertion into a wound cavity. The composite body includes a hydrophilic foam matrix. A first layer of polymer-based mesh is disposed at a first position within the foam matrix and at least a second layer of polymer-based mesh is disposed at a second position within the foam matrix in spaced apart opposing relation to the first layer of polymer-based mesh. The first layer of polymer-based mesh and the second layer of polymer-based mesh are in sandwiching relation to a foam core.

In accordance with another aspect, the present invention provides a method for treating a cavity or tunneling wound. The method includes providing a wound dressing having a composite body of predetermined length wherein the wound dressing includes a hydrophilic foam matrix, a first layer of polymer-based mesh disposed at a first position within the foam matrix and at least a second layer of polymer-based mesh disposed at a second position within the foam matrix in spaced apart opposing relation to the first layer of polymer-based mesh. The first layer of polymer-based mesh and the second layer of polymer-based mesh are disposed in sandwiching relation to a foam core. A distal portion of the wound dressing is inserted into the wound towards a base of the wound. The wound dressing is retained in the wound for a time sufficient to allow at least partial healing of the wound while wicking fluid away from the base of the wound. The wound dressing is withdrawn at least partially away from the base of the wound as the wound heals. The foam holds excess exudate and pulls non-viable tissue from the wound as the dressing is removed.

While the invention will be described in connection with the preferred embodiment, it is understood that the invention is not intended to be so limited. On the contrary, it is intended to cover all alternatives, modifications, and equivalents as may be included within the spirit and scope of the invention as defined by the appended claims.

### BRIEF DESCRIPTION OF THE DRAWINGS

FIG. 1 is a schematic illustration of an exemplary elongate wound dressing in accordance with the present invention;

FIG. 2 is a schematic cross-sectional view taken along line 2-2 in FIG. 1;

FIG. 3 is a partial cut-away surface view of the exemplary elongate wound dressing taken generally along line 3-3 of FIG. 2, illustrating a mesh layer in embedded relation to a foam body;

FIG. 4 is a schematic illustration showing insertion of the exemplary elongate wound dressing with a convenient size tool into a wound cavity; and

FIG. 5 is a schematic of an exemplary processing line for forming the layered composite structure of the exemplary elongate wound dressing.

### DETAILED DESCRIPTION OF THE PREFERRED EMBODIMENT

Reference will now be made to the drawings, wherein like elements are designated by like reference numerals throughout the various views.

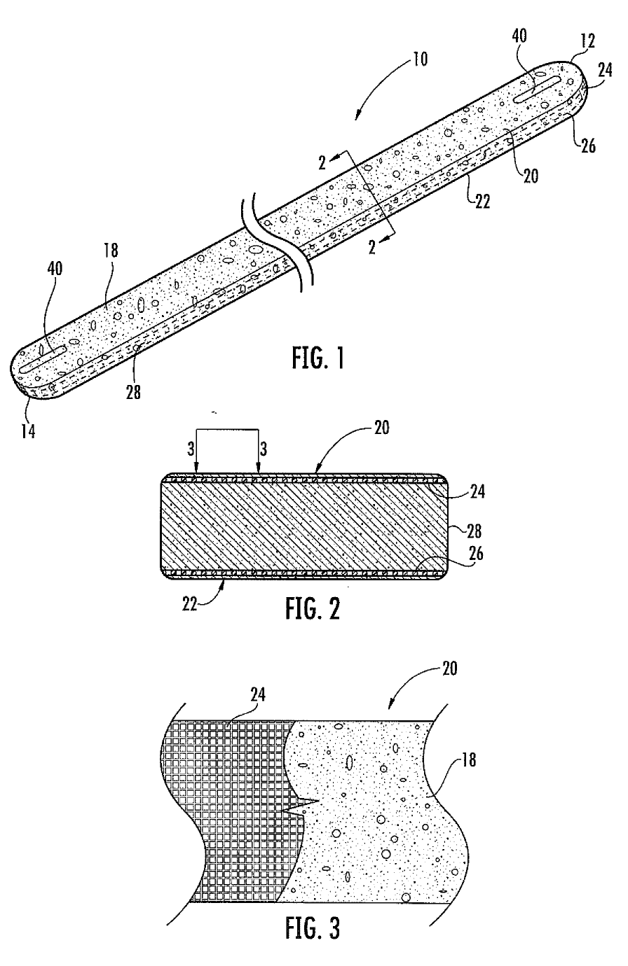

FIG. 1 illustrates a wound dressing 10 of elongate construction suitable for insertion into a wound cavity in a manner as will be described further hereinafter. As illustrated, the exemplary wound dressing 10 includes a first end 12 and a second end 14. The first end 12 and the second end 14 may be either similar or dissimilar relative to one another. In the illustrated and potentially preferred construction, the first end 12 and the second end 14 are generally rounded to facilitate smooth insertion into a wound cavity. However, other configurations such as square faces, chevron profiles and the like may also be used if desired.

As shown, the wound dressing 10 is of a generally extended length construction with a length dimension substantially greater than the width dimension and thickness dimension. By way of example only, in one exemplary construction corresponding to FIGS. 1 and 2, the wound dressing 10 is of a ribbon configuration. In one potentially preferred embodiment, the dressing 10 has a length dimension of about 14 inches, a width dimension of about 0.4 inches and a thickness dimension of about 0.2 inches in a dry state. As best illustrated in FIG. 2, such a dressing has a generally rectangular cross section. Of course, it is to be understood that the length and/or width of the dressing may be varied substantially. While a ribbon configuration is illustrated and may be desirable for many uses, it is likewise contemplated that a rope configuration having a polygonal or rounded cross section may be used as desired.

Referring jointly to FIGS. 1-3, the exemplary wound dressing 10 is formed from a polymer-based foam as will be described further hereinafter. The foam defines a flexible substantially unitary block foam body 18 having a first or upper face 20 and a second or lower face 22 disposed in substantially opposing relation to one another. A first mesh layer 24 is disposed in at least partially embedded relation within the foam body 18 in substantially adjacent relation to the upper face 20. A second mesh layer 26 is disposed in at least partially embedded relation within the foam in substantially adjacent relation to the lower face 22. In this configuration, a foam core 28 is sandwiched between the first mesh layer 24 and the second mesh layer 26. The foam core 28 incorporates a network of interconnected open cells adapted to transmit fluid between the cells.

The first mesh layer 24 and the second mesh layer 26 may be of similar or dissimilar character. By way of example only and not limitation, in one potentially preferred embodiment the first mesh layer 24 and the second mesh layer 26 are each formed from a surgical implant grade filament from a thermoplastic polymer resin of the polyester family such as polyethylene terephthalate (PET) or the like. However, it is contemplated that other bio-compatible mesh materials which do not degrade after insertion may be used if desired. Such materials may include polypropylene, nylon and the like.

The material forming the first mesh layer 24 and the second mesh layer 26 is preferably characterized by interstitial openings of dimension sufficient to permit uncured foam precursor material to flow through the openings and around the filaments. By way of example only, it is believed that mesh formed from multi-filament polyester fiber elements with interstitial openings of about 0.5 mm to about 2 mm in width may be useful for a relatively wide range of foam precursor compositions. However, larger or smaller openings may be used if desired. In this regard, in general, larger diameter openings may be useful for use with high viscosity foam precursor materials, while smaller openings may be useful with low viscosity foam precursor materials. The first mesh layer 24 and the second mesh layer 26 preferably each have a thickness of about 0.1 mm to about 1 mm and more preferably about 0.2 mm to about 0.4 mm.

Preferably, the foam 18 substantially encapsulates and physically bonds to the fiber elements of the first mesh layer 24 and the second mesh layer 26. However, it is likewise contemplated that outwardly facing surfaces of the first mesh layer 24 and/or the second mesh layer 26 may remain at least partially uncovered by the foam. According to one potentially desirable practice, each of the mesh layers may be disposed at a depth of about 0.1 mm to about 2 mm, and more preferably about 0.1 mm to about 1 mm below the adjacent outer surface of the foam body 18. However, it is likewise contemplated that the first mesh layer and/or the second mesh layer may be positioned substantially deeper within the dressing if desired. In the illustrated and potentially preferred embodiment, the first mesh layer 24 and the second mesh layer 26 extend substantially to the edges of the dressing 10. That is, the mesh layers 24, 26 are fully coextensive with the length and width dimensions of the dressing. However, it is likewise contemplated that the mesh layers may extend less than the full length and/or width of the dressing 10 if desired. If desired, one or more additional layers of mesh or other material (not shown) may be disposed within the foam core at intermediate positions between the first mesh layer 24 and the second mesh layer 26 so as to segment the foam core 28 into two or more sections. By way of example only, and not limitation, such intermediate materials may be useful in providing additional dimensional stability to the dressing.

Regardless of whether or not intermediate layers of non-foam material are present within the foam core 28, it is believed that the collective thickness of the first mesh layer 24 and the second mesh layer 26 and any non-foam intermediate layers should be at a level in the range of about 0.5% to about 15% of the thickness of the foam disposed between the first mesh layer 24 and the second mesh layer 26. As will be described further hereinafter, despite such small relative thicknesses, the presence of the sandwiching first mesh layer 24 and second mesh layer 26 provides substantial improvement to the strength and wicking characteristics of the dressing 10.

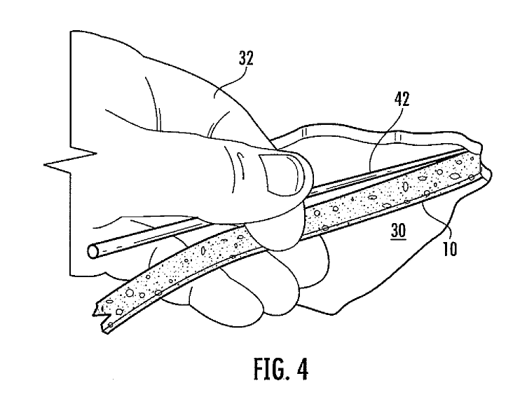

As best illustrated through reference to FIG. 4, the first end 12 of the wound dressing 10 may be inserted into a wound cavity 30 being treated such that it is embedded beneath the skin surface of the patient. In this orientation, the second end projects outwardly away from the entrance to the wound cavity such that it is accessible by a user 32. Of course, the selection of which end of the wound dressing 10 to insert into the wound cavity is discretionary with the user. For purposes of description, the end inserted into the wound cavity will be referred to as the distal end while the end extending outwardly which remains accessible by a user shall be referred to as the proximal end.

Referring jointly to FIGS. 1 and 4, in the exemplary illustrated construction, the exemplary wound dressing 10 includes apertures 40 defining openings extending through the thickness dimension of the dressing between the upper face 20 and the lower face 22. The apertures 40 are disposed in relatively close proximity to the first and second ends of the wound dressing. In the illustrated construction, the apertures 40 are in the form of slits having a major axis extending generally along the length dimension of the wound dressing 10 with the major axis in substantially centered relation relative to the width dimension of the wound dressing 10. However, it is likewise contemplated that other geometries including rounded or polygonal holes and the like arranged in centered or off-centered relation to the width dimension of the dressing the may be used if desired. Moreover, while apertures 40 are illustrated as being present at both ends of the wound dressing, a single aperture positioned proximate to a single end, or additional apertures along the length or width, may likewise be used if desired. Alternatively, the apertures 40 may be eliminated entirely if desired.

In the illustrated exemplary embodiment, the user 32 may use an insertion tool 42 (FIG. 4) such as a disposable long cotton swab or a probe having a forked end or other configuration adapted to engage the aperture proximate the distal end of the wound dressing 10. By advancing the insertion tool 42 into the wound cavity, the wound dressing 10 is correspondingly advanced until reaching the base of the wound cavity or other situs as may be desired. If desired, the insertion tool may include graduated markings to provide the user with an indication of insertion depth. Once a desired insertion depth has been achieved, the insertion tool 42 may be withdrawn from the wound cavity 16 thereby leaving the wound dressing in place. In this position, the wound dressing 10 may absorb and provide wicking action, wherein liquid wound exudate passes along the length of the dressing toward the proximal end thereof. An additional advantage of the described rope dressing is that the aperture accommodates many different suitable tools 42 for the convenience of the user.

As will be appreciated, while the wound dressing 10 is illustrated as having an elongated ribbon or rope-like geometry with a length dimension substantially greater than the width dimension, virtually any other geometry with embedded spaced mesh layers may likewise be used. By way of example only and not limitation, such geometries may include substantially rectangular, square or other polygonal shapes. Still other exemplary geometries may include cylindrical, conical, or frustro-conical shapes including such shapes as described in relation to U.S. Pat. No. 7,022,890 to Sessions (incorporated by reference).

As noted previously, the exemplary wound dressing 10 is a foam block body in which the sandwiching mesh layers 24, 24 are embedded. The foam forming the body is preferably a hydrophilic polymer-based foam of the type typically used for wound dressings. In this regard, polyurethane foams may be particularly desirable. By way of example only, and not limitation, potentially desirable polyurethane foams and methods of preparing such foams are described in U.S. Pat. Nos. 5,064,653 and 5,916,928 both to Sessions et al. the teachings of which are incorporated by reference as if fully set forth herein. Optionally, the foam may incorporate an anti-microbial agent. Exemplary anti-microbial agents include silver metal, silver alloys and silver salts such as silver nitrate. In this regard, the inclusion of silver metal in an amount of about 0.1% to about 2% by weight based on of the foam may be particularly preferred for some applications.

According to a potentially preferred practice, the foam is formed from the reaction of water with isocyanate-capped polyurethane prepolymers as will be known to those of skill in the art. The amount of prepolymer in the reactant composition used to prepare the hydrophilic foam composition typically depends on its isocyanate functionality and the degree of crosslinking desired in the final foam product. In general, the greater the isocyanate functionality, the greater the degree of cross-linking in the cured foam product. Typically, the reactant composition will include from about 20% to about 60% by weight prepolymer. Preferably the reactant composition will include from about 45% to about 50% by weight of the prepolymer.

The reactant composition forming the foam may, if desired, further include a hydrophilic agent which is incorporated into the foam composition to enhance absorption of external liquid, such as wound exudate, and to retain such liquid in the foam composition. The hydrophilic agent incorporated into the foam composition is believed to absorb fluid from the wound to assist thickening of the blood, i.e., it serves as a hemostat. Absorption of exudate by the hydrophilic agent, and the subsequent swelling of the agent results in the removal of inflammatory exudates and particles that would otherwise hinder tissue repair or cause eschar formation. Necrotic debris and bacteria are likewise removed as autolysis, i.e. chemical debridement, is stimulated. Suitable superabsorbent polymers include sodium and aluminum salts of starch, grafted copolymers of acrylates and acrylamides, and combinations thereof, as well as polyacrylate salts. Of course, other absorbent materials may be used in combination with such highly absorbent polymers. When such agents are employed, either alone or in combination, the resulting foam composition desirably has the ability to hold at least about three times its weight in liquid. In the preferred embodiment, the resulting foam composition will have the ability to tightly hold at least about three times its weight in fluid. As used herein “tightly held” or “tightly bound” liquid means the relative amount of liquid retained by the sample after compression.

The amount of hydrophilic agent used and the type of agent, in terms of its fluid uptake, that may be satisfactorily used to make the foam composition is not critical, but is, instead, dependent on the intended application of the resulting foam composition. However, the amount of hydrophilic agent utilized should not be so great as to undesirably reduce the strength of the foam composition or result in a loss of polymer from the foam, although some loss of hydrophilic agent may be tolerated without adversely affecting the ability of the foam to absorb external liquids. The amount of hydrophilic agent employed in the reactant composition will also depend on the absorbency of the material used. As previously indicated, it is preferable that a sufficient amount of hydrophilic agent be employed so that the resulting foam composition is capable of absorbing at least about three times its weight in external liquid. Typically this can be achieved by including from about 5 wt. % to about 20 wt. % hydrophilic agent in the reactant composition.

The reactant composition of this invention may further include an adjuvant; preferably, a water-soluble adjuvant. The adjuvant is releasably carried by the resulting foam composition for subsequent release to a chosen situs of application. Release of the adjuvant occurs in the presence of an external liquid, such as wound exudate, which is preferentially absorbed by the foam composition. Absorption of the external liquid causes at least a portion of the adjuvant to be released.

It will be appreciated by those skilled in the art that not all of the liquid adjuvant is necessarily released (or need it be) in the presence of the external fluid. However, a sufficient amount of adjuvant must be released in order to achieve the desired result. To that end, it will be appreciated that the efficacy of the adjuvant is realized upon its release from the foam composition to the wound cavity.

Prior to curing, the adjuvant serves as a plasticizer for the reactant composition. It extends the curing time of the composition thereby allowing it to be more thoroughly mixed and formed. Once cured, the foam composition is softened by the adjuvant, allowing the foam to be more pliable and more easily applied to the skin surface or other surface of choice. Additionally, the adjuvant may be somewhat hygroscopic lending further to the hydrophilic nature of the foam composition.

Adjuvants suitable for use in the foam composition of the present invention are mono-, di- and polyhydric alcohols. Preferably the adjuvants are water soluble so that they may be readily released from the composition upon contact of the foam composition with an external liquid. It is also preferred that the adjuvant be compatible with therapeutic or other agents which may be carried by the adjuvant for subsequent delivery to the situs of application. Suitable adjuvants include water soluble alcohols, including monols, diols and polyhydric alcohols. Examples of monols include ethyl alcohol and isopropyl alcohol. Exemplary of suitable diols are propylene glycol, polyethylene glycol, and polypropylene glycol. Exemplary of suitable polyhydric alcohols are glycerin, 1,2,4-butanetriol, trimethylolpropane, pentaerythritol, and sorbitol. In general, the molecular weight of the alcohols should be less than about 1000. Mixtures of alcohols can likewise be used. Glycerin may be a particularly preferred adjuvant because it has the attributes of a medicament, cosmetic, or therapeutic agent. Various additional medicaments, cosmetics, and therapeutic agents may, if desired, be carried with the adjuvant and released with it to the desired situs. This release thus allows the transmission of such therapeutic or other agents carried in the adjuvant to the area of application outside the foam composition, further assisting in the beneficial treatment of the wound.

The amount of adjuvant included in the reactant composition should preferably be sufficient to impart softness and pliability to the foam composition and be capable of delivering a therapeutic agent or the like, if included, to the environment of application. However, the volume of adjuvant should not be so great as to weaken or gel the composition. Generally, it has been found that the amount of adjuvant in the reactant composition should be from about 5 wt. % to about 30 wt. % of the reactant composition.

A wetting agent optionally may be included in the reactant composition to provide more uniform wetability of the resulting foam. The wetting agent also aids in controlling the cell size of the foam and in the reticulation of the final foam. Wetting agents suitable for use include non-ionic surfactants. Examples of materials that may be used as the wetting agent, either alone or in admixture, include block copolymers of ethylene oxide and propylene oxide, ethoxylated sorbitan fatty acid esters, glycerol esters, polyglycerol esters, and silicone fluids as will be well known to those of skill in the art. Generally, the amount of wetting agent should be from about 1% to about 10% by weight of the reactant composition, preferably from about 5% to about 7% by weight. The wetting agent should not react with the foam composition or any component of the foam formulation to create difficulties during foam formation or to adversely affect the desired characteristics of the foam composition in use or while being stored.

As will be appreciated by those of skill in the art, water is used in the initiation of the foaming reaction. It should be appreciated that the source of the water for the foaming reaction is not critical. The water so required may be provided as a separate component of the reactant composition, or, for example, it may be provided by one of the other components of the reactant composition. By way of illustration, and not in limitation, the required water may be provided with an aqueous-based cosmetic which may be incorporated into the foam composition. The type of water used is likewise not critical. However, for medical applications, purified water such as deionized or distilled water may be used. Saline solutions may also be used satisfactorily.

It will be appreciated that the relative proportion of prepolymer, adjuvant and hydrophilic agent, if the latter two are included in the reactant composition, can be varied over wide ranges in order to prepare a hydrophilic foam composition having the desired release and exchange characteristics previously described, while likewise providing a foam composition that is aesthetically satisfactory, insofar as its oilyness, touch, appearance and general feel.

As will be appreciated, while a potentially desirable foam product may be manufactured using an isocyanate-capped prepolymer, such prepolymers are exemplary only. Accordingly, it is contemplated that virtually any prepolymers yielding foams suitable for introduction into a human body may be satisfactorily employed.

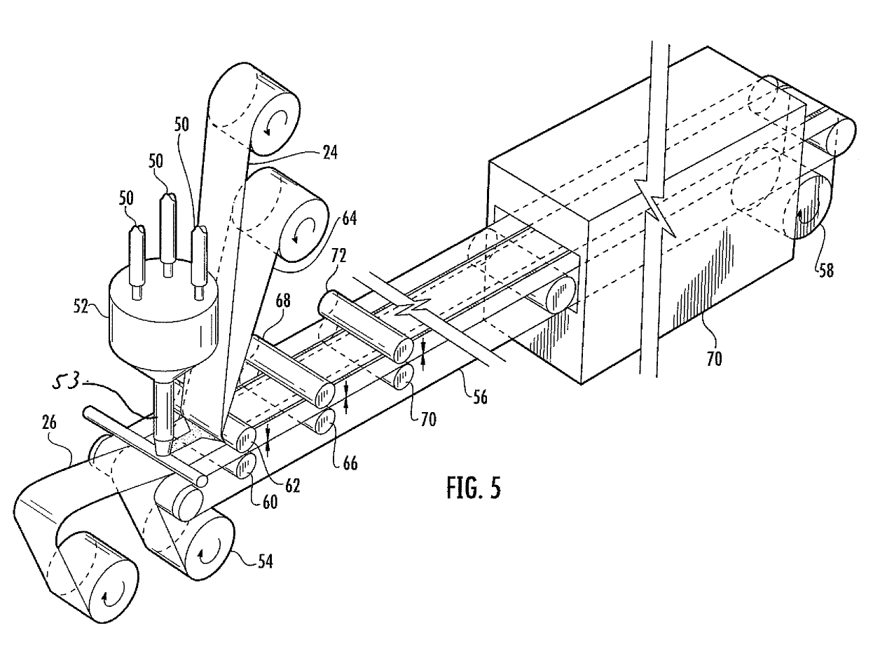

Turning to FIG. 5, a process is illustrated for producing a foam block composite with layers of embedded mesh. Once this composite is formed, the wound dressing 10 may be cut out and slits or other openings may be introduced to form the apertures 40. In the illustrated process, the raw material constituents of the foam such as adjuvant (or organic phase), prepolymer, and aqueous phase are transferred via inlet tubes 50 to a suitable reaction vessel 52 for combination and reaction. The reaction vessel 52 merely serves to mix the reactants sufficiently such that they will react to form the reaction product. The reaction vessel 52 is preferably equipped with speed-controllable mixing paddles to blend the phases and a temperature control means for controlling the temperature of the reactants.

The mixing speed of the vessel 52 and its temperature are preferably set to a predetermined level as a variance in either parameter will affect the properties of the resulting foam. Generally, the predetermined levels are dependent on the flow rates of each component and more specifically on the combined flow rate. For example, if the mixer revolutions per minute (rpm) is too low, inadequate mixing of the reactants results. If the mixer rpm is too high, the heat build up due to the high setting increases the reaction rate of the reactants, thereby effecting the subsequent processing of the reaction product.

The temperature of the mixer is generally kept lower than the temperature of the reactants because the reaction itself is exothermic. If the temperature is too high, the reaction will proceed at a much higher rate, thereby effecting subsequent processing of the foam, and also shortening the cure time. Excessive temperatures can also cause an imbalance in carbon dioxide generation and polymerization which may result in a nonuniform product.

After the mixing process is completed, the reaction product may be discharged from vessel 52 through a nozzle 53. During the discharge of the reaction product, the mesh material defining the second mesh layer 26 in the wound dressing 10 (FIG. 2) and an underlying sacrificial backing layer 54 are fed onto a continuous conveyor 56 traveling below the nozzle 53. By way of example only, one potentially desirable sacrificial backing layer 54 that may be used is a silicone coated polystyrene sheet. However, any substantially impermeable material that may be easily removed from the final formed foam may be used. As shown, the reaction product from the vessel 52 is deposited on top of the mesh material defining the second mesh layer 26. During deposit, a portion of the substantially uncured reaction product passes through the openings in the second mesh layer 26 and is collected at the underlying sacrificial backing layer 54. In this regard, it will be understood that the second mesh layer 26, may float upwardly slightly as the reaction product collects across the sacrificial backing layer. However, such upward floating is limited by the tension from the take-up winder 58 located at the end of the process line.

In accordance with a potentially desired practice, the sacrificial backing layer 54 and the material forming the second mesh layer 26 move forward relative to the nozzle 53 at the same velocity as the conveyer 56. The flow rate of the reaction product from the nozzle 53 and the velocity of the sacrificial backing layer 54 are set to control the thickness and width of the resulting foam sheet. In this regard, the velocity of the backing layer 54 is directly proportional to the reaction time of the reaction product prior to compression by a first set of compression rollers 60, 62 and affects the nature of the reaction product.

By way of example only, the conveyer 56 may be designed so as to allow the velocity of the second mesh layer 26 and sacrificial backing layer 54 to vary from about 0.1 to about 11 feet per minute, with the rate at which the reaction product is deposited through nozzle 53 being within the range from about 0.1 to about 2.0 pounds per minute. According to one potentially desirable process where the reaction product leaves the reaction vessel at 90 degrees Fahrenheit after being mixed at 2500 rpm, the reaction product is deposited at a rate of approximately 0.7 pounds per minute and the backing layer 54 travels at a velocity of about 4 feet per minute.

After the reaction product is deposited from nozzle 53, but before the reaction product is subjected to its initial compression by rollers 60, 62, a sheet of mesh material forming the first mesh layer 24 is laid into the deposited reaction product in combination with an overlying cover layer 64 of substantially impermeable character. The cover layer 64 is preferably release coated, and is thus releasably adhered to the product. If desired, the cover layer 64 may be formed from the same material as the sacrificial backing layer. Accordingly, one potentially desirable cover layer that may be used is a silicone coated polystyrene sheet. However, any substantially impermeable material that may be easily removed from the final formed foam may be used.

Subsequent to, or simultaneously with, the introduction of the first mesh layer 24 and the cover layer 64, the formed composite is subjected to a compressive force which serves to control the thickness of the resulting foam sheet product. According to a potentially desirable practice, the first compression may take place as the reaction product takes on a cream state and begins to foam and rise. While it is contemplated that the composite may undergo only one compression, it is preferred that it undergo multiple compressions. The compressions are preferably accomplished on a continuous basis by passing the composite through a series of compression rollers 66, 68 and 70, 72 each of which defines a gap therebetween. The compression rollers compress and spread the creamed foam so as to effect a reduction in foam thickness of from about 5 to about 95 percent of the foam thickness just prior to compression. Reductions of that magnitude may be effected for each of a plurality of compressions. It will be appreciated by those skilled in the art that the number of compressions, degree of compression, and the timing of the compressions may be adjusted to achieve a desired final foam character. More specifically, the density, thickness, width, and appearance of the product will be affected. In order to determine the number and degree of compressions for the particular reaction product and processing conditions employed, a measurement of the foam thickness of the reactant product that has been removed from the conveyor just after each sequential compression may be taken after the foam has been allowed to rise to its fullest extent. This measurement may then be compared with a measurement taken of the thickness of the foam reaction product that has similarly been allowed to rise to its fullest extent without undergoing that compression. Such a comparison will allow an operator to determine both the number of compressions and the degree of compression needed to attain a foam having the desired final thickness.

In the illustrative embodiment of the invention, the initial compression preferably reduces the thickness of the reaction product by about 80 percent, and each subsequent compression reduces the thickness by about 40 percent. Compressing the composite in this manner results in a superior final foam product that will emerge having a specific, predetermined thickness. By way of example only, when the velocity of the substrate backing layer 54 is about 5 feet per minute, and the reaction product is deposited at a rate of 0.2 pounds per minute, it is preferred that the initial compression takes place within about 2 seconds after the reaction product leaves the nozzle. The second and third compressions, each of which compresses the foam about 40 percent, should then occur within 55 and 70 seconds, respectively, after the material has left the nozzle 53.

In accordance with a potentially desirable practice, final curing of the foam takes place at ambient conditions following compression without the introduction of heat. If desired, the composite exiting the final compression rolls may subsequently be subjected to drying means wherein moisture level within the foam is reduced to a predetermined level. Preferably the moisture level in the final foam product is about 10 percent or less by weight. Advantageously, drying is carried out using a hot air impingement dryer 70, with the air that is used for the dryer being first drawn through a particulate filter. According to one potentially desirable practice, the drying temperature is in the range from about 100 degrees Fahrenheit to about 175 degrees Fahrenheit and most preferably about 140 degrees Fahrenheit.

Following drying, the resultant composite may be collected in a jelly-roll arrangement about the take-up winder 58. Thereafter, sections of the resultant composite may be removed from the collection roll and cut to predetermined lengths and shapes as described. The backing layer 54 and the cover layer 64 may be removed before or after such cutting. Virtually any three-dimensional shape may be achieved by folding the composite and adjoining edges together by adhesives, laser butt welding or other suitable techniques as may be know to those of skill in the art. The resultant structure is then useable as a wound dressing providing absorption and high wicking capacity of wound fluids.

### Comparative Examples 1 and 2

Benefits associated with the present invention may be further understood through reference to the following non-limiting comparative examples evaluating wicking capacity.

According to the test method, polyurethane foam based wound dressings with and without embedded mesh sandwiching layers, but otherwise substantially identical in all respects were arranged with a first end held in submerged relation across the bottom of an elevated tray filled with saline solution. The opposing ends of the dressings were snaked over the edge of the tray and were placed in a beaker at an elevation below the tray. The beaker was covered without applying pressure to the dressings in order to reduce evaporation. The dressings were allowed to become saturated with the saline solution and to thereafter expel excess fluid into the beaker on a drop-wise basis. After 24 hours, the fluid level in the beaker was measured. In each test, two wound dressings of identical construction were arranged in parallel to one another from a common saline bath and fed into a common beaker to reduce statistical variability. In the dressings incorporating the mesh sandwiching layers, two substantially identical polyester mesh layers were used, corresponding to the arrangement in FIGS. 1 and 2. The mesh layers each had a thickness of about 0.2 mm with interstitial openings of about 1 mm×0.9 mm. The results are set forth in Table I below:

TABLE 1

| | Dressing Width | Dressing Length | Number of | Embedded | Total Wicked | Wicked Fluid per |
|---|---|---|---|---|---|---|
| Sample | Tested | Tested | Dressings | Sandwiching | Fluid | Dressing |
| No. | (cm) | (cm) | Tested | Mesh | (ml) | (ml) |
| 1 | 1.0 | 35.6 | Two | Yes | 700 | 350 |
| 2 | 1.0 | 35.6 | Two | No | 335 | 167.5 |

The result of these tests demonstrates a greater than 100% improvement in wicking capability as a result of incorporating the embedded sandwiching mesh.

### Comparative Examples 3-14

The procedures as outlined with respect to Examples 1 and 2 were repeated using various known commercial dressings adapted for use in deep wound cavities. Each of the dressings was cut to a common length of 12 inches (30.5 cm). Sample No. 11 was a tubular structure and was fed into a beaker at a height above the saline bath to prevent non-wicking siphoning action through the interior. Wicking is reported in grams due to the low volume of collected fluid and the enhanced precision of using a weigh-based measurement for such low volumes. The specific gravity of the test saline solution is estimated to be approximately 1.004. Thus, the reported weight in grams is substantially equivalent to the volume of collected fluid as measured in milliliters. The results of the wicking tests are set forth in Table 2 below.

TABLE 2

| | | Packaged | Packaged | Tested | Number of | Wicked Fluid Per |
|---|---|---|---|---|---|---|
| Sample | Commercial | Width | Length | Length | Dressings | Dressing |
| No. | Name | (cm) | (cm) | (cm) | Tested | (grams) |
| 3 | ACTICOAT | 2.0 | 30.5 | 30.5 | One | 0 |
| | ABSORBENT | | | | | |
| 4 | CALCICARE | 2.5 | 30.5 | 30.5 | One | 0 |
| 5 | FIBRACOL PLUS | 1.0 | 40.0 | 30.5 | One | 0 |
| 6 | MAXORB EXTRA | 2.5 | 30.5 | 30.5 | One | 0 |
| 7 | SEASORB | 2.5 | 44 | 30.5 | One | 0 |
| 8 | SILVERCEL | 2.5 | 30.5 | 30.5 | One | 0 |
| 9 | SORBSAN | 2.5 | 30.5 | 30.5 | One | 0 |
| 10 | TEGAGEN | 2.5 | 30.5 | 30.5 | One | 0 |
| 11 | HYDROFERA BLUE | 0.9 | 14 | 14 | One | 1.59 |
| 12 | AQUACEL AG | 2.0 | 40.0 | 30.5 | One | 3.92 |
| 13 | AQUACEL | 2.0 | 45.0 | 30.5 | One | 31.8 |
| 14 | ASKINA FOAM | 2.0 | 35.0 | 30.5 | One | 144.4 |
| | CAVITY | | | | | |

These results indicate that the present invention incorporating sandwiching mesh materials provides substantially improved wicking capability relative to a number of existing commercial products.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A wound dressing comprising:
a composite body of predetermined length adapted for insertion into a wound cavity, the composite body comprising; a hydrophilic foam matrix, a first layer of polymer-based mesh disposed at a first position within the foam matrix and at least a second layer of polymer-based mesh disposed at a second position within the foam matrix in spaced apart opposing relation to the first layer of polymer-based mesh, the first layer of polymer-based mesh and the second layer of polymer-based mesh disposed in sandwiching relation to a foam core, each of the first layer of polymer-based mesh and the second layer of polymer-based mesh extending substantially along the length of the composite body.

2. The wound dressing as recited in claim 1, wherein the composite body has a substantially ribbon configuration having a length dimension greater than a width dimension and wherein the width dimension is greater than a thickness dimension.

3. The wound dressing as recited in claim 2, wherein the composite body has at least one rounded end.

4. The wound dressing as recited in claim 3, wherein the composite body has two rounded ends.

5. The wound dressing as recited in claim 2, wherein the composite body includes at least one aperture extending through the thickness dimension.

6. The wound dressing as recited in claim 5, wherein said at least one aperture is a slit having a major axis extending substantially along the length dimension.

7. The wound dressing as recited in claim 1, wherein the foam core extends substantially uninterruptedly between the first layer of polymer-based mesh and the second layer of polymer-based mesh.

8. The wound dressing as recited in claim 1, wherein at least the first layer of polymer-based mesh is formed from a hydrophobic polymer.

9. The wound dressing as recited in claim 8, wherein the hydrophobic polymer is polyester.

10. The wound dressing as recited in claim 1, wherein each of the first layer of polymer-based mesh and the second layer of polymer-based mesh is formed from a hydrophobic polymer.

11. The wound dressing as recited in claim 10, wherein the hydrophobic polymer is polyester.

12. A wound dressing comprising:
an elongated composite body of predetermined length adapted for insertion into a wound cavity, wherein the composite body has a substantially ribbon configuration having a length dimension greater than a width dimension and wherein the width dimension is greater than a thickness dimension, the composite body having a first outer face and a second outer face projecting outwardly away from the first face, the composite body comprising; a hydrophilic open cell polyurethane foam matrix, a first layer of polymer-based mesh of hydrophobic character disposed within the foam matrix in substantially submerged relation adjacent to the first outer face and at least a second layer of polymer-based mesh of hydrophobic character disposed within the foam matrix in substantially submerged relation adjacent to the second outer face, the first layer of polymer-based mesh and the second layer of polymer-based mesh disposed in sandwiching relation to a foam core, the first layer of polymer-based mesh being substantially coextensive with the first outer face and the second layer of polymer-based mesh being substantially coextensive with the second outer face.

13. The wound dressing as recited in claim 12, wherein the composite body has at least one rounded end.

14. The wound dressing as recited in claim 13, wherein the composite body has two rounded ends.

15. The wound dressing as recited in claim 12, wherein the composite body includes at least one aperture extending through the thickness dimension.

16. The wound dressing as recited in claim 15, wherein said at least one aperture is a slit having a major axis extending substantially along the length dimension.

17. The wound dressing as recited in claim 12, wherein the first layer of polymer-based mesh of hydrophobic character is polyester.

18. The wound dressing as recited in claim 17, wherein the second layer of polymer-based mesh of hydrophobic character is polyester.

19. A wound dressing comprising:
a composite body of predetermined length adapted for insertion into a wound cavity, the composite body comprising; a hydrophilic foam matrix, a first layer of polymer-based mesh disposed at a first position within the foam matrix and at least a second layer of polymer-based mesh disposed at a second position within the foam matrix in spaced apart opposing relation to the first layer of polymer-based mesh, the first layer of polymer-based mesh and the second layer of polymer-based mesh disposed in sandwiching relation to a foam core, each of the first layer of polymer-based mesh and the second layer of polymer-based mesh extending substantially along the length of the composite body, wherein the wound dressing has increased liquid wicking capacity relative to a substantially identical structure lacking said first layer of polymer-based mesh and said second layer of polymer-based mesh.

20. A method of treating a cavity or tunneling wound, the method comprising the steps of:
(a) providing a wound dressing having a composite body of predetermined length, the wound dressing comprising:
(i) a hydrophilic foam matrix;
(ii) a first layer of polymer-based mesh disposed at a first position within the foam matrix; and
(iii) at least a second layer of polymer-based mesh disposed at a second position within the foam matrix in spaced apart opposing relation to the first layer of polymer-based mesh, the first layer of polymer-based mesh and the second layer of polymer-based mesh disposed in sandwiching relation to a foam core;
(b) inserting a distal portion of the wound dressing into the wound towards a base of the wound;
(c) retaining the wound dressing in the wound for a time sufficient to allow at least partial healing of the wound while wicking fluid away from the base of the wound; and
(d) withdrawing the wound dressing at least partially away from the base of the wound.
